(19) United States
(12) Patent Application Publication
(10) Pub. No.: US 2024/0423705 A1
(43) Pub. Date: Dec. 26, 2024

(54) COMBINATION THERAPY TOOL FOR ONCOLOGY MANAGEMENT

(71) Applicant: Covidien LP, Mansfield, MA (US)

(72) Inventor: Scott E.M. FRUSHOUR, Boulder, CO (US)

(73) Assignee: Covidien LP, Mansfield, MA (US)

(21) Appl. No.: 18/669,660

(22) Filed: May 21, 2024

Related U.S. Application Data
(60) Provisional application No. 63/522,941, filed on Jun. 23, 2023.

(51) Int. Cl. A61B 18/14; A61B 18/00; A61M 25/00

(52) U.S. Cl. CPC A61B 18/1492; A61B 2018/00029; A61B 2018/0022; A61B 2018/00577; A61M 25/0043

(57) ABSTRACT

A system for performing a surgical procedure includes a catheter including an ablation therapy delivering portion configured to deliver ablation therapy to an area of interest and an aperture configured to deliver a therapeutic agent to the area of interest, and a workstation operably coupled to the catheter, the workstation including an ablation therapy source operably coupled to the ablation therapy delivering portion of the catheter, a fluid source operably coupled to the aperture of the catheter, and a memory and a processor, the memory storing instruction, which when executed by the processor cause the processor to generate and deliver ablation therapy to the area of interest and deliver a therapeutic agent to the area of interest.

### CROSS-REFERENCE TO RELATED APPLICATIONS

This application claims the benefit of U.S. Provisional Patent Application Ser. No. 63/522,941, filed Jun. 23, 2023, the entire content of which is incorporated herein by reference.

### TECHNICAL FIELD

This disclosure relates to the field of ablation catheters and particularly to ablation catheters capable of delivering ablation therapy and a therapeutic agent to a target area.

### BACKGROUND

There are several commonly applied medical methods, such as endoscopic procedures or minimally invasive procedures, for treating various maladies affecting organs including the liver, brain, heart, lungs, gall bladder, kidneys, and bones. In the treatment of diseases such as cancer, localized ablation of tumor cells is effectuated using electromagnetic fields, chemical therapy, cryotherapy, for example. Such modalities may be delivered via an endoscope during a minimally invasive procedure and raise or lower the temperature of the tumor cells to a temperature that ablates or otherwise denatures the tumor cells. As can be appreciated, such therapy is applied in a manner to optimize the margin surrounding the lesion, destroying as many of the tumor cells as possible while minimizing damage to surround, healthy cells.

Diseases such as cancer are also treated using pharmaceutical agents delivered systemically. In some instances, following the systemic injection of the pharmaceutical agent, an ablation procedure is performed on the lesion in an effort to increase the probability that the entire lesion is destroyed. These treatment methods require two procedures to be performed; the systemic injection of the pharmaceutical agent and then the ablation procedure. As will be appreciated, improvements to the current methods of treatment of diseases such as cancer are desired.

### SUMMARY

In accordance with the disclosure, a system for performing a surgical procedure includes a catheter, the catheter including an ablation therapy delivering portion configured to deliver ablation therapy to an area of interest and an aperture configured to deliver a therapeutic agent to the area of interest, and a workstation operably coupled to the catheter, the workstation including an ablation therapy source operably coupled to the ablation therapy delivering portion of the catheter, a fluid source operably coupled to the aperture of the catheter, and a memory and a processor, the memory storing instructions, which when executed by the processor cause the processor to generate and deliver ablation therapy to the area of interest and deliver a therapeutic agent to the area of interest.

In aspects, the ablation therapy source may be selected from the group consisting of a microwave generator, a radio-frequency generator, a pulsed electric field energy generator, a cryogenic ablation source, a chemical ablation source, a high intensity focused ultrasound source, and a lithotripsy source.

In certain aspects, the system may include a therapeutic agent delivery tube disposed on an outer surface of the catheter, wherein a proximal end of the therapeutic agent delivery tube is operably coupled to the fluid source, wherein the therapeutic agent delivery tube extends longitudinally along the catheter in a distal direction and terminates at the aperture.

In other aspects, the catheter may define an interior portion, wherein a cooling channel is disposed within the interior portion of the catheter, the cooling channel operably coupled to the fluid source.

In certain aspects, the aperture may be defined through an outer surface of the catheter, wherein the aperture is in fluid communication with the cooling channel and permits fluid flowing within the cooling channel to be expelled through the aperture and into the area of interest.

In other aspects, the aperture may include a plurality of perforations defined through the outer surface of the catheter, wherein each perforation of the plurality of perforations is in fluid communication with the cooling channel.

In aspects, the catheter may include a balloon disposed about the ablation therapy delivering portion, the balloon configured to transition from a deflated configuration to an inflated configuration where an outer portion of the balloon abuts a portion of the area of interest.

In certain aspects, the cooling channel may extend into and out of the balloon, wherein the aperture is disposed distal of the balloon.

In other aspects, a bore may be defined through the cooling channel, the bore in fluid communication with the fluid source and permitting fluid to flow from the fluid source and into an interior portion of the balloon.

In certain aspects, the cooling channel may terminate at the aperture within an interior portion of the balloon.

In aspects, a lumen may be defined through the balloon, wherein the lumen is in fluid communication with the interior portion of the balloon, the lumen permitting fluid to flow from the interior portion of the balloon and into the area of interest.

In accordance with another aspect of the disclosure, a catheter includes an ablation therapy delivering portion disposed adjacent to a distal end of the catheter, wherein the ablation therapy delivering portion is operably coupled to an ablation therapy source, a therapeutic agent delivering portion disposed adjacent to the distal end of the catheter, wherein the therapeutic agent delivering portion is operably coupled to a fluid source and is configured to deliver a therapeutic agent to an area of interest, and a cooling channel disposed within an interior portion of the catheter.

In aspects, a therapeutic agent delivering tube may be disposed on an outer surface of the catheter, wherein a proximal portion of the therapeutic agent delivering tube is in fluid communication with the fluid source and a distal portion of the therapeutic agent delivering tube terminates at an aperture.

In certain aspects, an aperture may be defined through an outer surface of the catheter, wherein the aperture is in fluid communication with an interior portion of the catheter and the cooling channel.

In other aspects, the catheter may include a distal end cap, the distal end cap including a plurality of perforations extending into the interior portion of the catheter and configured to permit fluid within the interior portion of the catheter to flow into the area of interest.

In certain aspects, the catheter may include a movable cover slidably disposed on an outer surface of the catheter, the movable cover configured to transition from a closed position where the movable cover is disposed over the aperture and inhibits the flow of fluid through the aperture to an open position where the movable cover exposes the aperture to permit fluid to flow into the area of interest.

In accordance with another aspect of the disclosure, a method of performing a surgical procedure includes applying ablation therapy to an area of interest, the ablation therapy delivered by an ablation therapy delivering portion of a catheter, the ablation therapy delivering portion of the catheter operably coupled to an ablation therapy source, and delivering a therapeutic agent to the area of interest where the ablation therapy was applied, the therapeutic agent delivered by an aperture defined on a distal portion of the catheter, wherein the aperture is in fluid communication with a fluid source.

In aspects, delivering the therapeutic agent to the area of interest may include delivering the therapeutic agent by the aperture in fluid communication with a therapeutic agent delivery tube disposed on an outer surface of the catheter.

In certain aspects, the method may include cooling the ablation therapy delivering portion of the catheter using a cooling fluid flowing within an interior portion of the catheter.

In other aspects, the aperture may be in fluid communication with the interior portion of the catheter enabling the cooling fluid to flow through the aperture and into the area of interest, wherein the cooling fluid is the therapeutic agent.

### BRIEF DESCRIPTION OF THE DRAWINGS

Various aspects and embodiments of the disclosure are described hereinbelow with references to the drawings, wherein:

FIG. 1 is a schematic view of a navigation system in accordance with the disclosure;

FIG. 2 is a schematic view of a catheter provided in accordance with the disclosure shown operably coupled to the navigation system of FIG. 1;

FIG. 3 is a front, cross-sectional view of the catheter of FIG. 2;

FIG. 4 is a schematic view of the catheter of FIG. 2 shown delivering therapy to an area of interest;

FIG. 5 is a schematic view of another embodiment of a catheter in accordance with the disclosure shown operably coupled to the navigation system of FIG. 1;

FIG. 6 is a cross-sectional view of the catheter of FIG. 5;

FIG. 7 is a cross-sectional view of another embodiment of the catheter of FIG. 5 in accordance with the disclosure;

FIG. 8 is a cross-sectional view of yet another embodiment of the catheter of FIG. 5 in accordance with the disclosure;

FIG. 9 is a cross-sectional view of still another embodiment of the catheter of FIG. 5 in accordance with the disclosure;

FIG. 10 is a schematic view of the catheter of FIG. 5 shown delivering therapy to an area of interest;

FIG. 11 is a partial cross-sectional view of an embodiment of the catheter of FIG. 5 having a balloon shown in an inflated state in accordance with the disclosure;

FIG. 12 is schematic view of another embodiment of the catheter of FIG. 5 in accordance with the disclosure shown delivering therapy to an area of interest;

FIG. 13 is a partial cross-sectional view of an embodiment of the catheter of FIG. 5 having another embodiment of a balloon shown in an inflated state in accordance with the disclosure;

FIG. 14 is a schematic view of another embodiment of a catheter in accordance with the disclosure shown delivering therapy to an area of interest;

FIG. 15 is a cross-sectional view of the catheter of FIG. 14 shown with an aperture in a closed position;

FIG. 16 is a cross-sectional view of the catheter of FIG. 14 shown with the aperture in an open position;

FIG. 17 is a schematic view of another embodiment of a catheter in accordance with the disclosure;

FIG. 18 is a flow diagram of a method of performing a surgical procedure provided in accordance with the disclosure;

FIG. 19 is a perspective view of a robotic surgical system provided in accordance with the disclosure;

FIG. 20 is an exploded view of a drive mechanism of a catheter of the navigation system of FIG. 1; and

FIG. 21 is a schematic view of a computing system for use with the navigation system of FIG. 1 provided in accordance with the disclosure.

### DETAILED DESCRIPTION

The disclosure is directed to a system having a catheter operably coupled to an ablation therapy source and a fluid source. In embodiments the system may be utilized with a robotic surgical system. The catheter includes an ablation therapy delivering portion disposed adjacent to a distal end of the catheter and a therapeutic agent delivering portion disposed adjacent to the distal end of the catheter. The ablation therapy source may be, for example, a microwave generator, a radio-frequency generator, a pulsed electric field energy generator, a cryoablation source, a chemical ablation source, a steam source, a high intensity focused ultrasound source, or a lithotripsy source. In one embodiment, the catheter includes a therapeutic agent delivery tube or hose that is disposed on an outer surface of the catheter. The therapeutic agent delivery tube is in fluid communication with the fluid source having a proximal end operably coupled to the fluid source and the distal end terminating at an aperture disposed at the therapeutic agent delivering portion. In this manner, fluid supplied by the fluid source flows through the therapeutic agent delivery tube and is expelled from the aperture into an area of interest. The catheter may include a cooling channel disposed within an interior portion of the catheter. It is contemplated that the cooling fluid flowing through the cooling channel may be the same or different fluid expelled from the aperture (e.g., for example, saline or a therapeutic agent). As can be appreciated, by including both an ablation therapy delivering portion and a therapeutic agent delivering portion on the catheter enables treatment of the area of interest using both modalities without having to move or otherwise reposition the catheter. Additionally, treatment of the area of interest using both modalities may be completed during the same procedure, reducing the time required for each treatment, the expense of performing each treatment, and trauma to the patent.

In embodiments, the aperture of the therapeutic agent delivering portion may be defined through an outer surface of the catheter. In this manner, the aperture is in fluid communication with the cooling channel and interior portion of the catheter, permitting fluid within the interior portion of the catheter to flow into the area of interest. As can be appreciated, the fluid flowing within the cooling channel and the interior portion of the catheter may be the same as the therapeutic agent. Myriad cooling channel configurations may be utilized to both cool the ablation therapy delivering portion and deliver the therapeutic agent to the area of interest, such as for example, one inflow channel and one outflow channel, one inflow channel and two outflow channels, two inflow channels and two outflow channels, and a separate inflow/outflow channel concentrically interposed between an ablation therapy cable or antenna and a pair of outflow channels and an inflow channel. In embodiments, the catheter may include a distal end cap having a plurality of perforations permitting the flow of the therapeutic agent from the interior portion of the catheter to the area of interest.

The catheter may have a balloon disposed about the ablation therapy delivering portion that is configured to transition from a first, deflated position to a second, inflated position where an outer surface of the balloon abuts a portion of the area of interest. It is contemplated that the therapeutic agent delivery tube may extend into and out of the balloon, positioning the aperture distal of the balloon. The balloon may be inflated using a separate fluid from the therapeutic agent, or in embodiments, the therapeutic agent delivery tube may include one or more bores permitting the flow of the therapeutic agent into an interior portion of the balloon to cause the balloon to inflate. In another embodiment, the therapeutic agent delivery tube may terminate at the aperture within the interior portion of the balloon. In this manner, the therapeutic agent flows from the aperture and into the interior portion of the balloon to cause the balloon to inflate. The balloon may include one or more lumens permitting the flow of the therapeutic agent from the interior portion of the balloon into the area of interest.

It is contemplated that a plurality of pulsed electric field electrodes may be disposed on the outer surface of the balloon, such that when the balloon is inflated, the plurality of pulsed electric field electrodes abut a portion of the area of interest. One or more lumens may be defined through the outer surface of the balloon to permit the flow of the therapeutic agent from the interior portion of the balloon to the area of interest. It is envisioned that the one or more lumens may be disposed concentric with the plurality of pulsed electric field electrodes or may be disposed at any location on the outer surface of the balloon.

During operation, it is determined whether the body cavity in which the distal end of the catheter is disposed is solid (e.g., for example fluid filled) or air-filled (e.g., for example, an airway of the lungs). If the body cavity is solid, ablation therapy is delivered to the area of interest, after which the therapeutic agent is delivered to the area of interest subject to the ablation therapy. If the body cavity is air-filled, the therapeutic agent is delivered to the area of interest followed by the application of ablation therapy. In embodiments, the area of interest may be initially flooded with saline to promote coupling of the ablation therapy delivering portion to the area of interest. It is contemplated that catheter may be flushed with saline to expel any remaining therapeutic agent from the catheter and ensure the delivery of the desired dose of therapeutic agent.

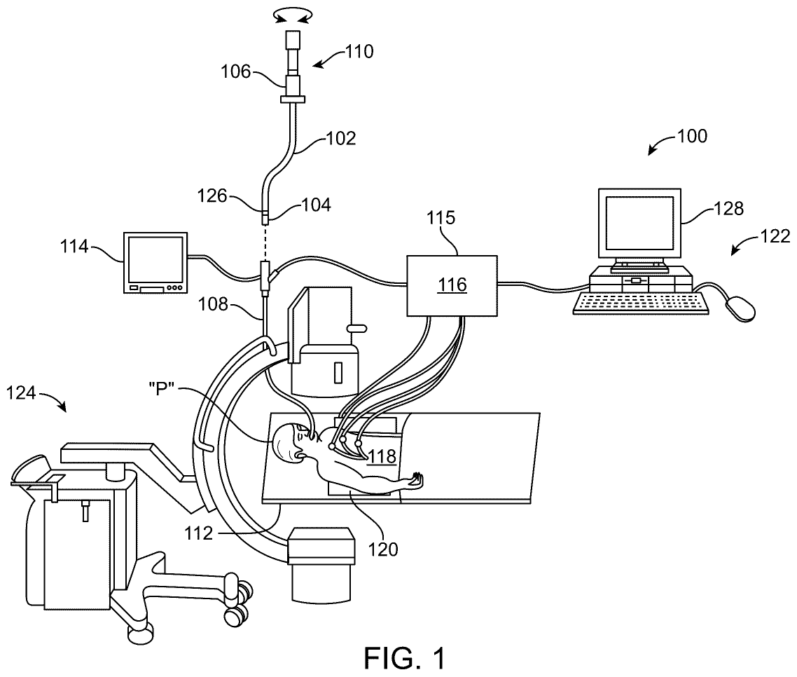

With reference to FIG. 1, a navigation system facilitating navigation of a medical device through a luminal network and to an area of interest is illustrated and generally identified by reference numeral 100. The navigation system 100 includes a catheter 102 and a guide assembly 106. In one embodiment, the catheter 102 is inserted into a bronchoscope 108 for access to a luminal network of the patient P. In this manner, the catheter 102 may be inserted into a working channel of the bronchoscope 108 for navigation through a patient's luminal network, such as for example, the lungs. The catheter 102 may itself include imaging capabilities via an integrated camera of optics component 109 and therefore, a separate bronchoscope 108 is not strictly required. A locatable guide (LG) 110 (a second catheter), including a sensor 104 may be inserted into the catheter 102 and locked into position such that the sensor 104 extends a desired distance beyond a distal tip of the catheter 102. As can be appreciated, the position and orientation of the sensor 104 relative to a reference coordinate system, and thus the distal portion of the catheter 102, within an electromagnetic field can be derived. Catheter guide assemblies 106 are currently marketed and sold by Medtronic PLC under the brand names SUPERDIMENSION® Procedure Kits, ILLUMISITE™ Endobronchial Procedure Kit, ILLUMISITE™ Navigation Catheters, or EDGE® Procedure Kits, and are contemplated as being usable with the disclosure.

Continuing with FIG. 1, the system 100 generally includes an operating table 112 configured to support a patient P and monitoring equipment 114 coupled to the bronchoscope 108 or catheter 102 (e.g., for example, a video display for displaying the video images received from the video imaging system of the bronchoscope 108 or the catheter 102), a locating or tracking system 115 including a tracking module 116, a plurality of reference sensors 118 and a transmitter mat 120 including a plurality of incorporated markers, and a workstation 122 having a computing device 128 including software and/or hardware used to facilitate identification of a target, pathway planning to the target, navigation of a medical device to the target, and/or confirmation and/or determination of placement of the catheter 102, or other suitable device therethrough, relative to the target.

A six degrees-of-freedom electromagnetic locating or tracking system 115, or other suitable system for determining position and orientation of a distal portion of the catheter 102, is utilized with the LG 110, as will be described in further detail hereinbelow, for performing registration of a detected position of the sensor 104 and a 3D model generated from a CT, CBCT, or MRI image scan. The tracking system 115 is configured for use with the LG 110 and particularly with the sensor 104. As described hereinabove, the LG 110 and the sensor 104 are configured for insertion through the catheter 102 into the patient P's airways (either with or without the bronchoscope 108) and are selectively lockable relative to one another via a locking mechanism (not shown).

With continued reference to FIG. 1, the transmitter mat 120 is positioned beneath the patient P. The transmitter mat 120 generates an electromagnetic field around at least a portion of the patient P within which the position of the plurality of reference sensors 118 and the sensor 104 can be determined with the use of the tracking module 116. A second electromagnetic sensor 126 may also be incorporated into the end of the catheter 102. The second electromagnetic sensor 126 may be a five degree-of-freedom sensor or a six degree-of-freedom sensor. One or more of the reference sensors 118 are attached to the chest of the patient P. Registration is generally performed to coordinate locations of the 3D model and 2D images from the planning phase, with the patient P's airways as observed through the bronchoscope 108 and allow for the navigation phase to be undertaken with knowledge of the location of the sensor 104.

Registration of the patient P′s location on the transmitter mat 120 may be performed by moving the sensor 104 through the airways of the patient P. In this manner, data pertaining to locations of the sensor 104, while the LG 110 is moving through the airways, is recorded using the transmitter mat 120, the reference sensors 118, and the tracking system 115. A shape resulting from this location data is compared to an interior geometry of passages of a 3D model, and a location correlation between the shape and the 3D model based on the comparison is determined, e.g., for example, utilizing the software on the computing device 128. In addition, the software identifies non-tissue space (e.g., for example, air filled cavities) in the 3D model. The software aligns, or registers, an image representing a location of the sensor 104 with the 3D model and/or 2D images generated from the 3D model, which are based on the recorded location data and an assumption that the LG 110 remains located in non-tissue space in a patient P's airways. Alternatively, a manual registration technique may be employed by navigating the bronchoscope 108 with the sensor 104 to pre-specified locations in the lungs of the patient P, and manually correlating the images from the bronchoscope 108 to the model data of the 3D model.

Although generally described with respect to EMN systems using EM systems, the instant disclosure is not so limited and may be used in conjunction with flexible sensors, such as for example, fiber-bragg grating sensors, inertial measurement units (IMU), ultrasonic sensors, without sensors, or combinations thereof. It is contemplated that the devices and systems described herein may be used in conjunction with robotic systems such that robotic actuators drive the catheter 102 or bronchoscope 108 proximate the target.

In accordance with aspects of the disclosure, the visualization of intra-body navigation of a medical device (e.g., for example a biopsy tool or a therapy tool), towards a target (e.g., for example a lesion) may be a portion of a larger workflow of a navigation system. An imaging device 124 (e.g., for example, a CT imaging device, such as for example, a cone-beam computed tomography (CBCT) device, including but not limited to Medtronic plc's O-arm™ system) capable of acquiring 2D and 3D images or video of the patient P is also included in the particular aspect of system 100. The images, sequence of images, or video captured by the imaging device 124 may be stored within the imaging device 124 or transmitted to the computing device 128 for storage, processing, and display. In embodiments, the imaging device 124 may move relative to the patient P so that images may be acquired from different angles or perspectives relative to the patient P to create a sequence of images, such as for example, a fluoroscopic video. The pose of the imaging device 124 relative to the patient P while capturing the images may be estimated via markers incorporated with the transmitter mat 120. The markers are positioned under the patient P, between the patient P and the operating table 112, and between the patient P and a radiation source or a sensing unit of the imaging device 124. The markers incorporated with the transmitter mat 120 may be two separate elements which may be coupled in a fixed manner or alternatively may be manufactured as a single unit. It is contemplated that the imaging device 124 may include a single imaging device or more than one imaging device.

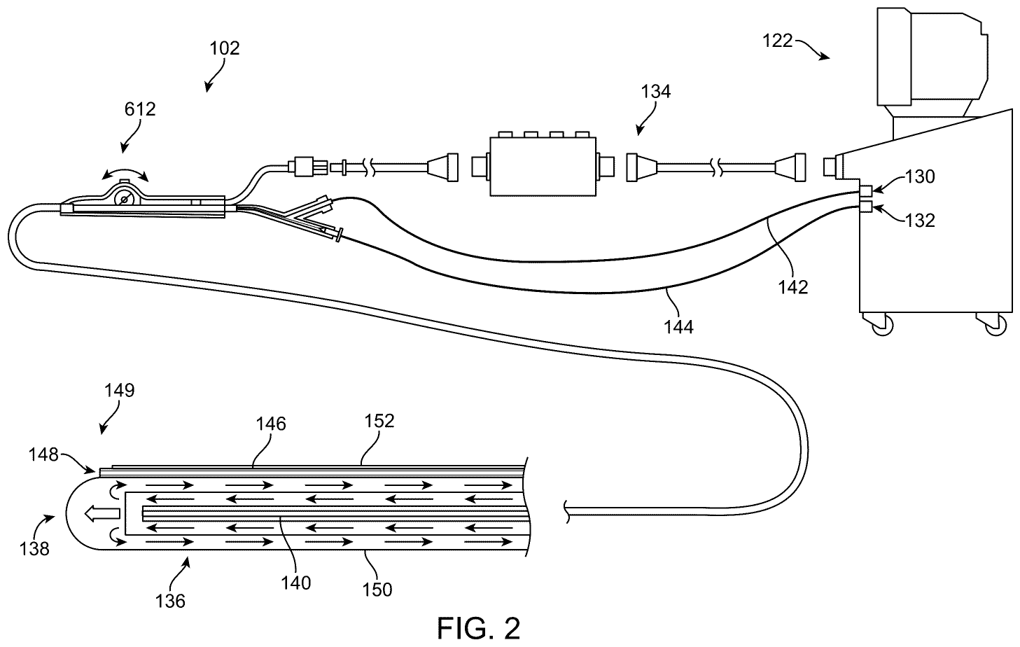

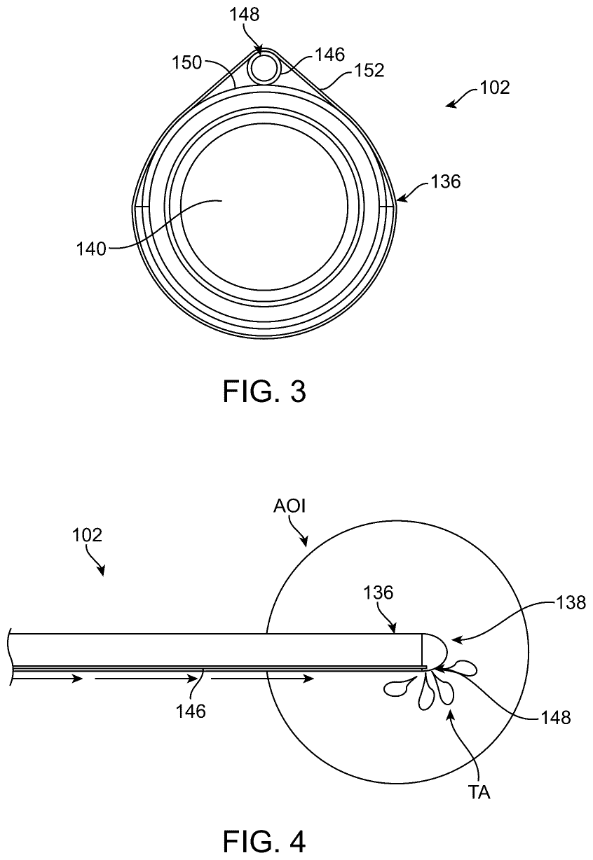

With additional reference to FIGS. 2-4, the workstation 122 includes an ablation therapy source 130 and a fluid source 132. The ablation therapy source 130 and the fluid source 132 may be formed of one or more separate sources, may be integrated into a single source, or may be disposed internal to or remote from the workstation 122 without departing from the scope of the disclosure. In embodiments, the ablation therapy source 130 may be disposed within the workstation 122 and the fluid source 132 may be disposed external to the workstation 122 or vice versa. The ablation therapy source 130 may be any ablation therapy source capable of delivering ablation therapy, such as for example, a microwave generator, a radio frequency (RF) generator, a pulsed electric field energy generator, a cryogen source, a chemical source, an ultrasonic generator, a steam source, a high intensity focused ultrasound source, or a lithotripsy source. The ablation therapy source 130 is operably coupled to the catheter 102 by an ablation therapy cable or hose 134, which in turn, is operably coupled to an ablation therapy delivering portion 136 disposed adjacent to or on a distal end 138 of the catheter 102. In embodiments, the ablation therapy delivering portion 136 may include, for example, a microwave antenna 140, one or more RF electrodes (not shown), one or more ultrasound transducers (not shown), a balloon (FIG. 11), and/or one or more needles (not shown).

The fluid source 132 is operably coupled to the catheter 102 via one or more hoses or tubes, such as for example, a first hose 142 and a second hose 144. In this manner, a cooling fluid may be delivered to the ablation therapy delivering portion 136 using the first hose 142 and returned to the fluid source 132 using the second hose 144. In embodiments, the first hose 142 may include a supply channel and a return channel to both supply and return cooling fluid to and from the ablation therapy delivering portion 136 and the second hose 144 may be utilized to deliver a therapeutic agent “TA”, such as for example, a chemotherapy or an immunotherapy fluid, which may include a molecularly targeted agent, to the target area, as will be described in further detail hereinbelow. It is envisioned that the cooling fluid and the therapeutic agent may be the same or different fluid. In embodiments, the fluid source 132 may deliver saline or other fluid capable of promoting coupling of the ablation therapy delivering portion 136 to the target tissue and/or promoting the flow of ablation therapy to and within the target tissue. It is envisioned that the fluid source 132 may deliver two separate fluids to be delivered to the target area. In this manner, the fluid source 132 may initially deliver a therapeutic agent to the target area and thereafter, the fluid source 132 may deliver saline or another suitable fluid for flushing the therapeutic agent from the catheter 102, or vice versa. It is envisioned that the therapeutic agent may be a heat sensitive agent, transitioning from one state to another, such as for example, from a fluid to a gel and vice versa. In embodiments, the therapeutic agent may be pre-heated or otherwise heated before being delivered to the target area or area of interest, or in embodiments, may be pre-cooled or otherwise cooled before being delivered to the target or area of interest. It is contemplated that the fluid source 132 may be operably coupled to a heater or cooler or other suitable device (not shown) configured to raise or lower a temperature of the therapeutic agent before being delivered to the area of interest.

Continuing with FIGS. 2-4, a therapeutic agent delivery tube 146 is disposed on and extends along an outer portion of the catheter 102 in a proximal to distal direction, which terminates at an aperture 148 adjacent the distal end 138 of the catheter 102, forming a therapeutic agent delivering portion 149 of the catheter 102. The therapeutic agent delivery tube 146 is in fluid communication with the fluid source 132. In this manner, the therapeutic agent flows through the therapeutic agent delivery tube 146 in a distal direction and is expelled from the aperture 148, which in turn, delivers the therapeutic agent to the target area. Although generally illustrated as being disposed adjacent to the distal end 138 of the catheter 102, it is envisioned that the aperture 148 may be disposed at any suitable location on the outer surface of the catheter 102 depending upon the design needs of the catheter 102 and the procedure being performed.

It is envisioned that the therapeutic agent delivery tube 146 may be disposed on and coupled to an outer surface 150 of the catheter 102 using any suitable means, such as for example, adhesives and/or fasteners. In embodiments, the therapeutic agent delivery tube 146 is interposed between the outer surface 150 of the catheter 102 and a sheath, insulating layer, or other layer 152 disposed about the outer surface 150 of the catheter. It is contemplated that the therapeutic agent delivery tube 146 may be disposed within a channel or groove (not shown) formed within the outer surface 150 of the catheter 102, or in embodiments, may be disposed within a bore defined within the body of the catheter 102 that extends along the length of the catheter 102 in a proximal to distal direction. In this manner, the therapeutic agent delivery tube 146 may be formed integral to the catheter 102 (e.g., for example, the bore may be formed within and through the catheter 102 rather than being a separate component. As can be appreciated, the cross-sectional profile of the therapeutic agent delivery tube 146 may be any suitable profile, such as for example, circular, hexagonal, racetrack, elliptical, square, or rectangular. In one non-limiting embodiment, the therapeutic agent delivery tube 146 may include an elliptical cross-sectional profile, which reduces an amount the therapeutic agent delivery tube 146 extends in a radial direction from the catheter 102 as compared to a circular, square, or other profile. As can be appreciated, having both the ablation therapy delivering portion 136 and the therapeutic agent delivery portion (via the aperture 148) disposed on the catheter 102 enables treatment of the area of interest using both modalities without having to move or otherwise reposition the catheter 102. Additionally, treatment of the area of interest using both modalities may be completed during the same procedure, reducing the time required for each treatment, the expense of performing each treatment, and trauma to the patent.

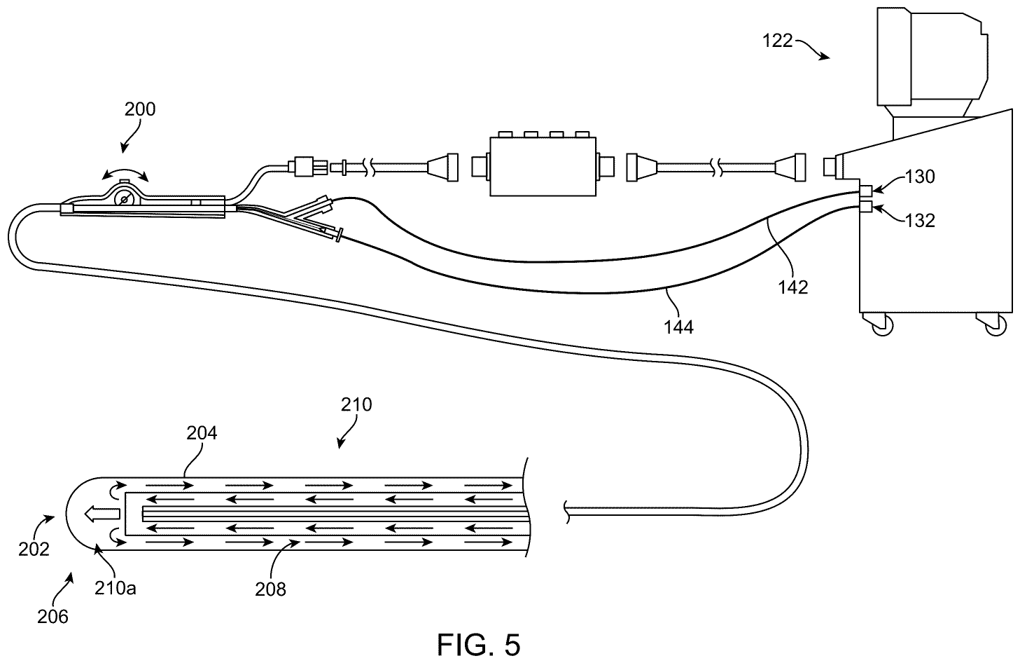

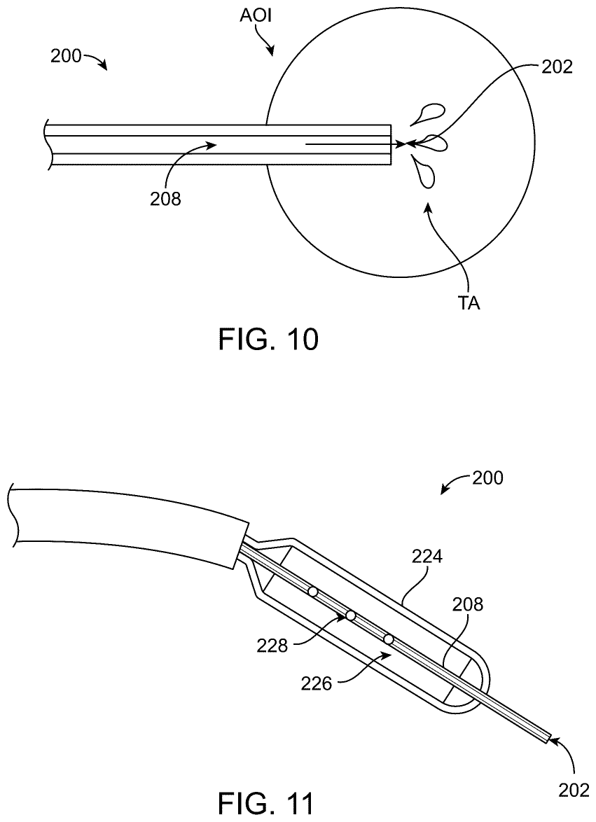

Turning to FIGS. 5-10, another embodiment of a catheter for use with system 100 is illustrated and generally identified by reference numeral 200. The catheter 200 is substantially similar to the catheter 102 described hereinabove and therefore, only the differences therebetween will be described herein in the interest of brevity. In lieu of a therapeutic agent delivery tube 146 disposed on the outer surface 150 of the catheter 102, the catheter 200 includes an aperture 202 defined through the outer surface 204 on the distal end 206 of the catheter 200. The aperture 202 is in fluid communication with cooling channels 208 disposed within an interior portion 210a of the ablation therapy delivery portion 210 of the catheter 200. In this manner, as fluid is supplied to the cooling channels 208 by the fluid source 132 of the workstation 122, a portion of the fluid is expelled from the aperture 202 and into the target area or area of interest. As can be appreciated, the fluid supplied by the fluid source 132 may be any suitable fluid, such as for example, saline or a therapeutic agent.

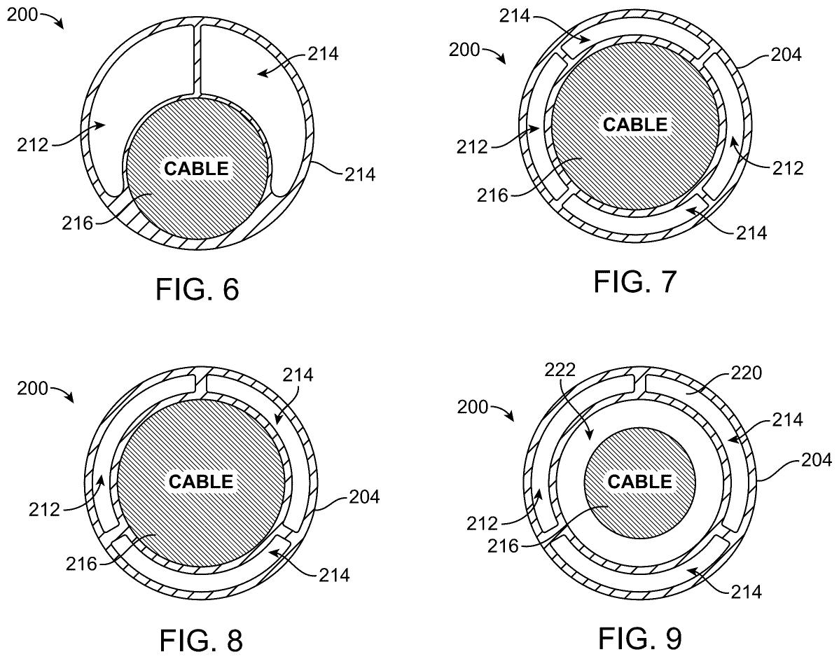

It is envisioned that the catheter 200 may include myriad channel designs, any one of which may be utilized depending upon the design needs of the catheter 200 or the procedure being performed. In this manner, the catheter 200 may include an inflow channel 212 and an outflow channel 214 partially enveloping a cable 216 coupling the ablation therapy source 130 to the ablation therapy delivering portion 210 (FIG. 6). In this manner, fluid is supplied by the fluid source 132 to the inflow channel 212 via one of the first and second hoses 142, 144. A portion of the fluid supplied by the fluid source 132 is expelled through the aperture 202 and to the area of interest while the remaining fluid is returned to the fluid source 132 via the remaining one of the first and second hoses 142, 144. Although generally described as delivering only a portion of the fluid supplied to the ablation therapy delivering portion 210, it is envisioned that most or all of the fluid supplied to the ablation therapy delivering portion 210 may be expelled through the aperture 202 and into the area of interest, although it is contemplated that any suitable amount of fluid may be delivered to the area of interest depending upon the procedure being performed.

Although generally illustrated as being offset from a center of the catheter 200, it is envisioned that the cable 216 may be disposed at any suitable location within the catheter 200. In one non-limiting embodiment, the cable 216 is coaxially aligned with the catheter 200. The catheter 200 may include two inflow channels 212 and two outflow channels 214 (FIG. 7) encircling the cable 216, may include one inflow channel 212 and two outflow channels 214 (FIG. 8) encircling the cable 216, may include one inflow channel 212 and two outflow channels 214 in a first concentric ring 220 disposed about the cable 216 and a combination inflow/outflow channel 222 interposed between the first concentric ring 220 and the cable 216 (FIG. 9), for example. It is envisioned that one of the outflow channels 214 may be utilized to circular cooling fluid within the catheter 200 and the remaining outflow channel 214 may be utilized to deliver a fluid to the area of interest via the aperture 202, such as for example, a therapeutic agent or saline.

With additional reference to FIG. 11 it is contemplated that the catheter 200 may include a balloon 224 disposed about the ablation therapy delivering portion 210. The balloon 224 is transitionable from a first, collapsed or deflated configuration to a second, expanded or inflated configuration. An interior portion 226 of the balloon 224 is in fluid communication with the fluid source 132, where a fluid, such as for example, saline, may be supplied to the interior portion 226 and cause the balloon 224 to transition from the first, deflated configuration to the second, inflated configuration. In embodiments, the cooling channel 208 may be perforated or otherwise include one or more bores 228 defined therein to permit a portion of the cooling fluid or therapeutic agent to enter the interior portion 226 and transition the balloon 224 from the first, deflated position, to the second, inflated position while enabling the cooling fluid or therapeutic agent to be expelled from the aperture 202 and into the area of interest. It is envisioned that the cooling channel 208 may be imperforate and the interior portion 226 of the balloon may be in fluid communication with one or more of the inflow or outflow channels 212, 214 (FIGS. 6-9). In this manner, the therapeutic agent may be delivered to the area of interest separate from the fluid utilized to inflate the balloon 224.

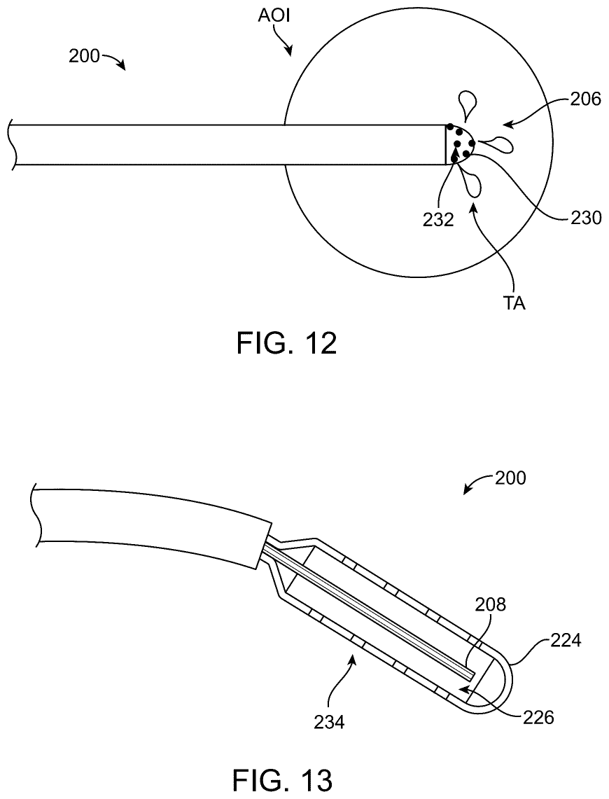

With further reference to FIGS. 12 and 13, it is envisioned that the catheter 200 may include a distal end cap 230 disposed on the distal end 206. A plurality of perforations 232 is defined through the distal end cap 230 with each perforation of the plurality of perforations 232 being in fluid communication with the cooling channel 208. In this manner, fluid supplied by the fluid source 132 is expelled through the plurality of perforations 232 and into the area of interest. It is envisioned that the plurality of perforations 232 may each have the same diameter or one or more perforations of the plurality of perforations 232 may include a different diameter than the remaining perforations. As can be appreciated, each perforation of the plurality of perforations 232 may include any suitable diameter without departing from the scope of the present disclosure, which may increase or decrease the flowrate and quantity of the fluid supplied to the area of interest.

In embodiments, the balloon 224 may include one or more lumens 234 (FIG. 13) defined therethrough and in fluid communication with the interior portion 226. In this manner, fluid received within the interior portion 226 is permitted to flow through the lumens 234 and into the target tissue. It is contemplated that the cooling channel 208 may terminate within the interior portion 226 of the balloon 224 such that fluid flowing within the cooling channel 208 may flow into the interior portion 226 of the balloon 224 and cause the balloon 224 to transition from the first, deflated position to the second, inflated position. Although generally described as terminating within the interior portion 226 of the balloon, it is envisioned that the cooling channel 208 may extend through the balloon 224 in a manner similar to that described hereinabove with respect to FIG. 11 without departing from the scope of the present disclosure.

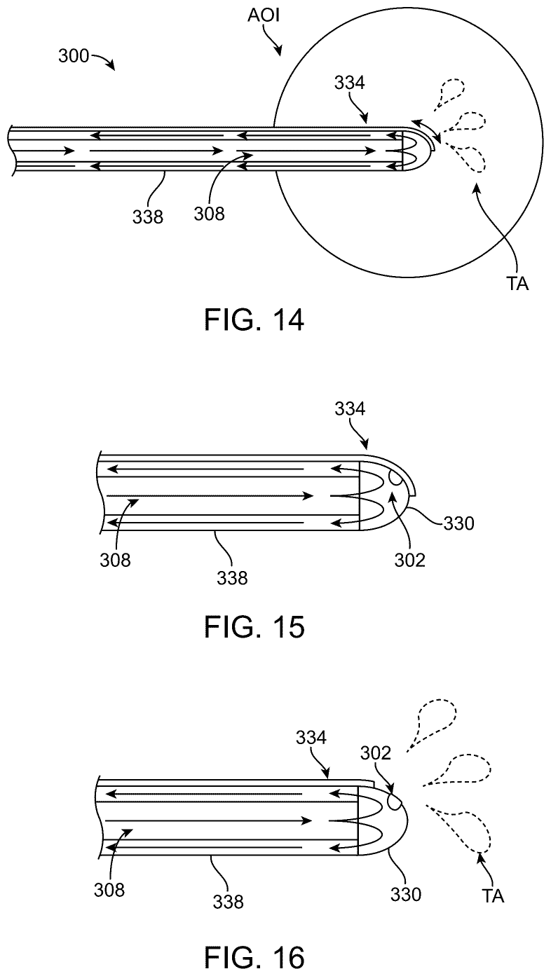

Turning to FIGS. 14-16, another embodiment of a catheter for use with the system 100 is illustrated and generally identified by reference numeral 300. The catheter 300 is substantially similar to the catheter 200 described hereinabove, and therefore, only the differences therebetween will be described in detail hereinbelow.

The catheter 300 includes a valve or movable cover 334 disposed on the distal end 306 of the catheter 300 which selectively inhibits the flow of fluid through the aperture 302 and into the target tissue. In this manner, the movable cover 334 is movable from a first, closed position (FIG. 15) where movable cover 334 covers or otherwise is disposed over the aperture 302 and inhibits the flow of fluid through the aperture 302 to a second, open position (FIG. 16) where the movable cover 334 exposes or otherwise opens the aperture 302 to permit fluid to be expelled therefrom. It is envisioned that the movable cover 334 may be manually or automatically actuated via a control handle of the catheter 300, via the workstation 122 (FIG. 5), or any other suitable means. It is envisioned that the movable cover 334 may be a strip of resilient material disposed within a channel or groove (not shown) formed within an outer surface 338 of the catheter 300. In embodiments, the movable cover 334 may be a sleeve formed from a resilient material that is configured to circumferentially cover the outer surface 338 of the catheter 300. In this manner, the movable cover 334 may conform to an outer profile of the catheter 300 (e.g., for example, increase or decrease in diameter due to a corresponding increase or decrease in diameter of the catheter 300), where proximal movement of the movable cover 334 causes a distal portion of the movable cover 334 to expand to accommodate the increase in diameter of the catheter 300 from the distal end cap 330 to the outer surface 338 and expose the aperture 302. It is contemplated that the movable cover 334 may be formed from a shape memory material formed from a metallic material such as for example, nitinol, a ceramic, or a polymer. In embodiments, the movable cover 334 may be a membrane formed from a pressure sensitive material where pressure exerted on the movable cover 334 by the fluid within the cooling channel 308 causes the movable cover 334 to act as a flap (e.g., for example, hingedly coupled to the catheter 300), act as a valve (e.g., for example, a self-scaling perforation), for example. It is envisioned that the movable cover 334 may be formed from a temperature sensitive material that contracts or expands in response to a change in temperature to either expose or close the aperture 302.

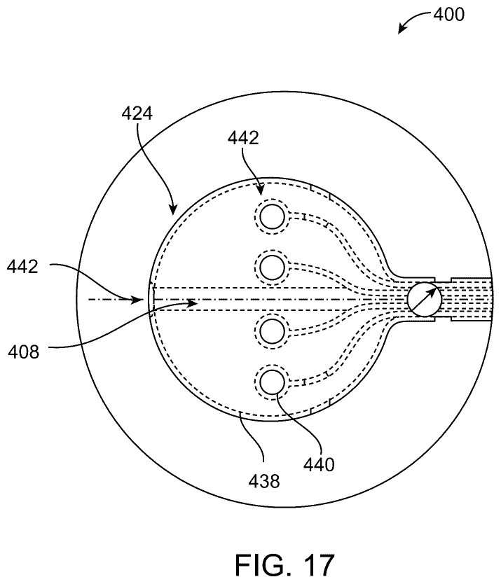

With reference to FIG. 17, another embodiment of a catheter for use with the system 100 is illustrated and generally identified by reference numeral 400. The catheter 400 is substantially similar to the catheter 200 described hereinabove except that the catheter 400 includes a plurality of pulsed electric field electrodes 440 disposed on an outer surface 438 of the balloon 424. As can be appreciated, the ablation therapy source 130 (FIG. 5) is a pulsed electric field energy generator that is operably coupled to each of the plurality of pulsed electric field electrodes 440. It is envisioned that the outer surface 448 of the balloon 424 may include one or more lumens 442 disposed adjacent to each electrode of the pulsed electric field electrodes 440, although it is contemplated that the lumens 442 may be disposed at any suitable location on the outer surface 438 of the balloon 424.

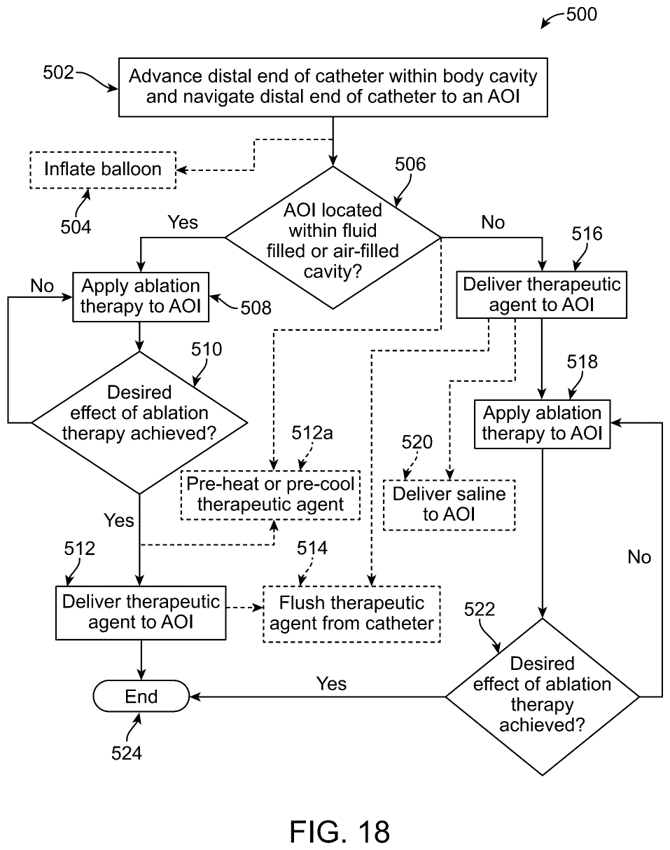

With reference to FIG. 18, a method of performing a surgical procedure is illustrated and generally identified by reference numeral 500. Initially, at step 502, the distal end 138, 206 or distal end cap 230, 330 is advanced into a body cavity of a patient and navigated to an area of interest (AOI). With the distal end 138, 206, or distal end cap 230, 330 located adjacent the area of interest (AOI), in step 504, if the catheter 102, 200, 300, 400 includes a balloon 234, 424, the balloon 234, 434 is transitioned from the first, deflated position to the second, inflated position such that an outer surface of the balloon 234, 434 abuts or otherwise contacts a portion of the area of interest (AOI). In step 506, it is determined if the area of interest (AOI) is located in a fluid filled cavity (e.g., for example, a solid) or an air-filled cavity (e.g., for example, an airway of the lung). If it is determined that the area of interest (AOI) is located within a fluid filled cavity, in step 508, excitation or ablation therapy is generated by the ablation therapy source 130 and applied to the area of interest (AOI) adjacent to the ablation therapy delivering portion 136, 210. As described hereinabove, the ablation therapy applied to the area of interest may be, for example, microwave ablation, RF ablation, cryoablation, chemical ablation, high intensity focused ultrasound, or lithotripsy. In step 510, it is determined if additional applications of ablation therapy are required, and if so, the method returns to step 508 where additional ablation therapy is applied to the area of interest (AOI) until the desired effect of the ablation therapy is achieved. In step 512, after the desired effect of ablation therapy is achieved, a therapeutic agent is supplied to the area of interest (AOI) via the fluid source 132 and the aperture 148, 202, 302 or the one or more lumens 234, 442 of the catheter 102, 200, 300, or 400. It is contemplated that the therapeutic agent may be heated or cooled before being supplied to the area of interest (AOI), and if required, the therapeutic agent may be pre-heated or pre-cooled in step 512a. Depending upon the procedure being performed and the therapeutic agent utilized, in step 514, the fluid source 132 may supply saline or another suitable fluid to flush or otherwise push any therapeutic agent remaining within the catheter into the area of interest (AOI), thereby ensuring that the entirety of the desired dose is delivered to the area of interest (AOI).

In step 516, if it determined that the area of interest (AOI) is located within an air-filled cavity, before applying the ablation therapy, a therapeutic agent is first supplied to the area of interest (AOI) via the fluid source 132 and the aperture 148, 202, 302 or the one or more lumens 234, 442 of the catheter 102, 200, 300, or 400. Thereafter, in step 518, excitation or ablation therapy is generated by the ablation therapy source 130 and applied to the area of interest (AOI) adjacent to the ablation therapy delivering portion 136, 210. In embodiments, saline or another suitable fluid may be supplied to the area of interest (AOI) via the fluid source 132 in step 520 to promote the transmission of the ablation therapy to the area of interest (AOI) and achieve the desired margin. In step 522, it is determined if additional applications of ablation therapy are required, and if so, the method returns to step 518 where additional ablation therapy is applied to the area of interest (AOI) until the desired effect of the ablation therapy is achieved. Once the desired effect of the ablation therapy is achieved, in step 524, therapy on the area of interest (AOI) is terminated. As can be appreciated, the above-described method may be performed as many times as necessary depending upon the procedure being performed and the number of areas of interest (AOI) that require treatment.

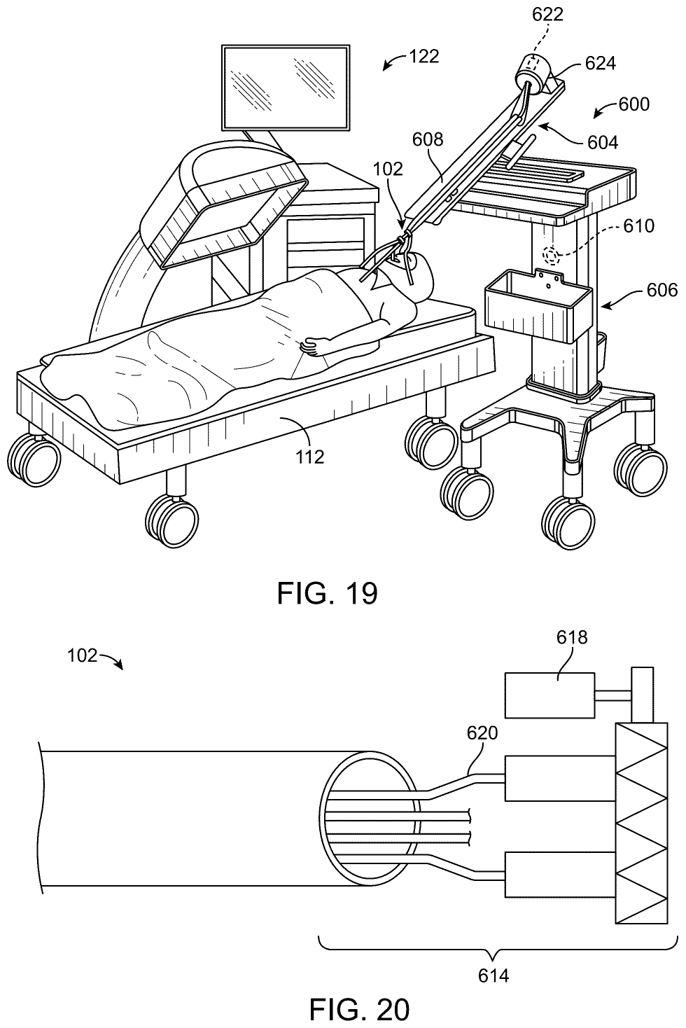

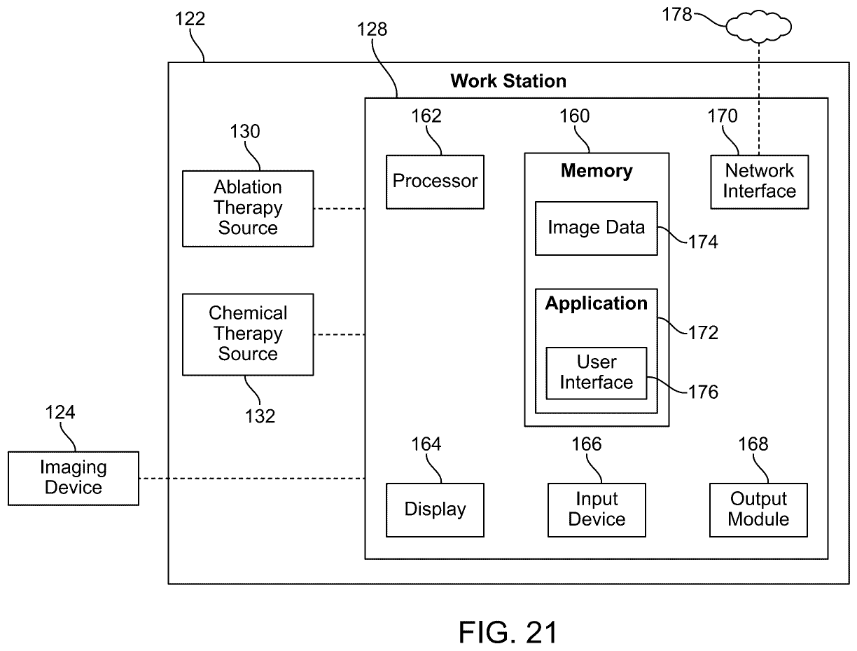

Turning to FIGS. 19 and 20, it is envisioned that the system 100 may include a robotic surgical system 600 having a drive mechanism 602 including a robotic arm 604 operably coupled to a base or cart 606, which may, in embodiments, be the workstation 122. The robotic arm 604 includes a cradle 608 that is configured to receive a portion of the catheter 102 thereon. The catheter 102 is coupled to the cradle 608 using any suitable means (e.g., for example, straps, mechanical fasteners, and/or couplings). It is envisioned that the robotic surgical system 600 may communicate with the catheter 102 via electrical connection (e.g., for example, contacts and/or plugs) or may be in wireless communication with the catheter 102 to control or otherwise effectuate movement of one or more motors (FIG. 20) disposed within the catheter 102 and receive images captured by a camera (not shown) associated with the catheter 102. In this manner, it is contemplated that the robotic surgical system 600 may include a wireless communication system 610 operably coupled thereto such that the catheter 102 may wirelessly communicate with the robotic surgical system 600 and/or the workstation 122 via Wi-Fi, Bluetooth®, for example. As can be appreciated, the robotic surgical system 600 may omit the electrical contacts altogether and may communicate with the catheter 102 wirelessly or may utilize both electrical contacts and wireless communication. The wireless communication system 610 is substantially similar to the network interface (FIG. 21) described hereinbelow, and therefore, will not be described in detail herein in the interest of brevity. As indicated hereinabove, the robotic surgical system 600 and the workstation 122 may be one in the same, or in embodiments, may be widely distributed over multiple locations within the operating room. It is contemplated that the workstation 122 may be disposed in a separate location and the display (FIG. 21) may be an overhead monitor disposed within the operating room.

As indicated hereinabove, it is envisioned that the catheter 102 may be manually actuated via cables or push wires, for example, or for example, may be electronically operated via one or more buttons, joysticks, toggles, actuators 612 (FIG. 2) operably coupled to a drive mechanism 614 disposed within an interior portion of the catheter that is operably coupled to a proximal portion of the catheter 102, although it is envisioned that the drive mechanism 614 may be operably coupled to any portion of the endoscope 102. The drive mechanism 614 effectuates manipulation or articulation of the distal end 138 (FIG. 2) of the catheter 102 in four degrees of freedom or two planes of articulation (e.g., for example, left, right, up, or down), which is controlled by two push-pull wires, although it is contemplated that the drive mechanism 614 may include any suitable number of wires to effectuate movement or articulation of the distal end 138 of the catheter 102 in greater or fewer degrees of freedom without departing from the scope of the present disclosure. It is contemplated that the distal end 138 of the catheter may be manipulated in more than two planes of articulation, such as for example, in polar coordinates, or may maintain an angle of the distal end 138 relative to the longitudinal axis of the endoscope while altering the azimuth of the distal end 138 of the catheter 102 or vice versa. In one non-limiting embodiment, the system 100 may define a vector or trajectory of the distal end 138 of the catheter 102 in relation to the two planes of articulation.

It is envisioned that the drive mechanism 614 may be cable actuated using artificial tendons or pull wires 616 (e.g., for example, metallic, non-metallic, and/or composite) or may be a nitinol wire mechanism. In embodiments, the drive mechanism 614 may include motors 618 or other suitable devices capable of effectuating movement of the pull wires 620. In this manner, the motors 618 are disposed within the catheter 102 such that rotation of an output shaft the motors 618 effectuates a corresponding articulation of the distal end 138 of the catheter 102.

Although generally described as having the motors 618 disposed within the catheter 102, it is contemplated that the catheter 102 may not include motors 618 disposed therein. Rather, the drive mechanism 614 disposed within the catheter 102 may interface with motors 622 disposed within the cradle 608 of the robotic surgical system 600. In embodiments, the catheter 102 may include a motor or motors 618 for controlling articulation of the distal end 138 of the catheter 102 in one plane (e.g., for example, left/null or right/null) and the drive mechanism 624 of the robotic surgical system 600 may include at least one motor 622 to effectuate the second axis of rotation and for axial motion. In this manner, the motor 618 of the catheter 102 and the motors 622 of the robotic surgical system 600 cooperate to effectuate four-way articulation of the distal end 138 of the catheter 102 and effectuate rotation of the catheter 102. As can be appreciated, by removing the motors 618 from the catheter 102, the catheter 102 becomes increasingly cheaper to manufacture and may be a disposable unit. In embodiments, the catheter 102 may be integrated into the robotic surgical system 600 (e.g., for example, one piece) and may not be a separate component.

With reference to FIG. 21, the computing device 128 of the workstation 122 may be any suitable computing device including a processor and a storage medium, wherein the processor is capable of executing instructions stored on the storage medium. In embodiments, the computing device 128 may include a memory 160, a processor 162, a display 164, and an input device 166. It is contemplated that the processor or hardware processor 132 may include one or more hardware processors. The computing device 128 may optionally include an output module 168 and a network interface 170. The memory 130 may store an application 172, patient data including image data 174, such as for example, CT data sets including CT images, fluoroscopic data sets including images and video, 3D reconstructions, navigation plans, and any other such data. Although not explicitly illustrated, the computing device 128 may include inputs, or may otherwise be configured to receive CT data sets, fluoroscopic images/video, and other data described herein. The application 172 may further include a user interface 176. The processor 162 may be coupled with the memory 160, the display 164, the input device 166, the output module 168, the network interface 170, and the imaging device 124. It is envisioned that the computing device 128 may be a stationary computing device, such as for example, a personal computer, or a portable computing device such as for example, a tablet computer. In embodiments, the workstation 122 may include a plurality of computing devices.

The memory 160 may include any non-transitory computer-readable storage media for storing data and/or software including instructions that are executable by the processor 162 and which control the operation of the workstation 122 and, in some embodiments, may also control the operation of the imaging device 124, the ablation therapy source 130, and the fluid source 132. The imaging device 124 may be used to capture a sequence of fluoroscopic images based on which the fluoroscopic 3D reconstruction is generated and to capture a live 2D fluoroscopic view according to this disclosure. In an embodiment, the memory 160 may include one or more storage devices such as for example, solid-state storage devices, e.g., for example, flash memory chips. Alternatively, or in addition to the one or more solid-state storage devices, the memory 160 may include one or more mass storage devices connected to the processor 162 through a mass storage controller (not shown) and a communications bus (not shown).

In embodiments, the application 172 may, when executed by the processor 162, cause the display 164 to present the user interface 176. The user interface 176 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view. The user interface 176 may be further configured to display the target mark in different colors depending on whether the medical device tip is aligned with the target in three dimensions.

Continuing with FIG. 22, the network interface 170 may be configured to connect to a network such as for example, the cloud 178, a local area network (LAN) consisting of a wired network and/or a wireless network, a wide area network (WAN), a wireless mobile network, a Bluetooth network, and/or the Internet. The network interface 170 may be used to connect between the workstation 122 and the imaging device 124. Network interface 170 may also be used to receive image data 174. It is envisioned that the input device 166 may be any device by which a user may interact with the workstation 122, such as for example, a mouse, keyboard, foot pedal, touch screen, and/or voice interface. The output module 168 may include any connectivity port or bus, such as for example, parallel ports, serial ports, universal serial busses (USB), or any other similar connectivity port known to those skilled in the art.

From the foregoing and with reference to the various figures, those skilled in the art will appreciate that certain modifications can be made to the disclosure without departing from the scope of the disclosure.

Although the description of computer-readable media contained herein refers to solid-state storage, it should be appreciated by those skilled in the art that computer-readable storage media can be any available media that can be accessed by the processor 162. That is, computer readable storage media may include non-transitory, volatile, and non-volatile, removable and non-removable media implemented in any method or technology for storage of information such as for example, computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 122.

What is claimed is:

1. A system, comprising:
a catheter including a portion configured to deliver ablation therapy to an area of interest and an aperture configured to deliver a therapeutic agent to the area of interest; and
a workstation operably coupled to the catheter, the workstation including:
an ablation therapy source operably coupled to the portion of the catheter;
a fluid source operably coupled to the aperture of the catheter; and
a memory and a processor, the memory storing instructions, which when executed by the processor cause the processor to:
generate and deliver ablation therapy to the area of interest; and
deliver a therapeutic agent to the area of interest.

2. The system according to claim 1, wherein the ablation therapy source is selected from the group consisting of a microwave generator, a radio-frequency generator, a pulsed electric field energy generator, a cryogenic ablation source, a chemical ablation source, a high intensity focused ultrasound source, and a lithotripsy source.

3. The system according to claim 1, further comprising a therapeutic agent delivery tube disposed on an outer surface of the catheter, wherein a proximal end of the therapeutic agent delivery tube is operably coupled to the fluid source, wherein the therapeutic agent delivery tube extends longitudinally along the catheter in a distal direction and terminates at the aperture.

4. The system according to claim 1, wherein the catheter defines an interior portion, wherein a cooling channel is disposed within the interior portion of the catheter, the cooling channel operably coupled to the fluid source.

5. The system according to claim 4, wherein the aperture is defined through an outer surface of the catheter, wherein the aperture is in fluid communication with the cooling channel and permits fluid flowing within the cooling channel to be expelled through the aperture and into the area of interest.

6. The system according to claim 4, wherein the aperture includes a plurality of perforations defined through an outer surface of the catheter, wherein each perforation of the plurality of perforations is in fluid communication with the cooling channel.

7. The system according to claim 4, wherein the catheter includes a balloon disposed about the portion, the balloon configured to transition from a deflated configuration to an inflated configuration where an outer portion of the balloon abuts a portion of the area of interest.

8. The system according to claim 7, wherein the cooling channel extends into and out of the balloon, wherein the aperture is disposed distal of the balloon.

9. The system according to claim 8, wherein a bore is defined through the cooling channel, the bore in fluid communication with the fluid source and permitting fluid to flow from the fluid source and into an interior portion of the balloon.

10. The system according to claim 7, wherein the cooling channel terminates at the aperture within an interior portion of the balloon.

11. The system according to claim 10, wherein a lumen is defined through the balloon, wherein the lumen is in fluid communication with the interior portion of the balloon, the lumen permitting fluid to flow from the interior portion of the balloon and into the area of interest.

12. A catheter, comprising:
a first portion disposed adjacent to a distal end of the catheter, wherein the first portion is operably coupled to an ablation therapy source;
a second portion disposed adjacent to the distal end of the catheter, wherein the second portion is operably coupled to a fluid source and is configured to deliver a therapeutic agent to an area of interest; and
a cooling channel disposed within an interior portion of the catheter.

13. The catheter according to claim 12, wherein a tube is disposed on an outer surface of the catheter, wherein a proximal portion of the tube is in fluid communication with the fluid source and a distal portion of the tube terminates at an aperture.

14. The catheter according to claim 12, wherein an aperture is defined through an outer surface of the catheter, wherein the aperture is in fluid communication with the interior portion of the catheter and the cooling channel.

15. The catheter according to claim 14, wherein the catheter includes a distal end cap, the distal end cap including a plurality of perforations extending into the interior portion of the catheter and configured to permit fluid within the interior portion of the catheter to flow into the area of interest.

16. The catheter according to claim 14, wherein the catheter includes a movable cover slidably disposed on an outer surface of the catheter, the movable cover configured to transition from a closed position where the movable cover is disposed over the aperture and inhibits the flow of fluid through the aperture to an open position where the movable cover exposes the aperture to permit fluid to flow into the area of interest.

17. A method, comprising:
applying ablation therapy to an area of interest, the ablation therapy delivered by a portion of a catheter operably coupled to an ablation therapy source; and
delivering a therapeutic agent to the area of interest where the ablation therapy was applied, the therapeutic agent delivered by an aperture defined on a distal portion of the catheter, wherein the aperture is in fluid communication with a fluid source.

18. The method according to claim 17, wherein delivering the therapeutic agent to the area of interest includes delivering the therapeutic agent by the aperture in fluid communication with a therapeutic agent delivery tube disposed on an outer surface of the catheter.

19. The method according to claim 17, further comprising cooling the portion of the catheter using a cooling fluid flowing within an interior portion of the catheter.

20. The method according to claim 19, wherein the aperture is in fluid communication with the interior portion of the catheter enabling the cooling fluid to flow through the aperture and into the area of interest, wherein the cooling fluid is the therapeutic agent.
